(19) United States
(12) Patent Application Publication
(10) Pub. No.: US 2023/0310794 A1
(43) Pub. Date: Oct. 5, 2023

(54) CALMING THERAPEUTIC PRESSURE-STRAP

(71) Applicant: Mary Beth Evans, San Jose, CA (US)

(72) Inventor: Mary Beth Evans, San Jose, CA (US)

(21) Appl. No.: 18/194,312

(22) Filed: Mar. 31, 2023

Related U.S. Application Data
(60) Provisional application No. 63/326,754, filed on Apr. 1, 2022.

(51) Int. Cl. A61M 21/02

(52) U.S. Cl. CPC A61M 21/02; A61M 2021/0022

(57) ABSTRACT

A therapeutic pressure strap for a mammal (animal or human). The strap comprises a flat laminated material with a textured elastic side and hook-and-loop second side, as well as a front end comprising a section of a complementary hook-and-loop material that can bind to the second side. The strap is configured so that when it is wound around the mammal's chest or trunk, the textured side resists slippage against the mammal's skin or fur, and the complementary material binds to the second side, producing a snug but reversible fit. The strap can be used to “swaddle” upset mammals and calm them without risk of overheating or causing panic due to overly restricting movement.

### CROSS REFERENCE TO RELATED APPLICATIONS

This application claims the priority benefit of U.S. provisional application 63/326,754, filed Apr. 1, 2022; the entire contents of which are incorporated herein by reference.

### BACKGROUND OF THE INVENTION

### Field of the Invention

This invention is in the field of garments and other devices to relieve anxiety.

### DESCRIPTION OF THE RELATED ART

Swaddling is known to be a way to calm mammals, such as human infants and animals. Previous workers, such as Blizzard, U.S. Pat. No. 8,291,867 have proposed pressure-applying garments for animals to relieve animal anxiety. Such garments tended to cover a large percentage of the animal's body. However, such prior art garments are cumbersome to use.

### BRIEF SUMMARY OF THE INVENTION

The invention is inspired, in part, by the insight that many mammals, including humans and animals, may be overwhelmed by the large amount of body surface area covered by prior art pressure garments, and feel overly constrained. Additionally, prior art calming garments could potentially lead to overheating, as well as being cumbersome to put on and take off. Thus, further improvements are desirable.

The invention is also inspired, in part, by the insight that similar considerations can apply to the pressure vests to calm humans; for example, consider humans who struggle with the sensory processing deficits associated with Autism. These individuals are often soothed with the application of deep pressure to organize their nervous system. Unfortunately, these prior art garments also tend to cover the entirety of the trunk, and often required third party assistance to don and doff.

The invention is further inspired by the insight that human individuals on the Autism Spectrum often have communication difficulties, and such individuals may not be able to express their need to be extracted from such prior art pressure garments. This can create harm by further increasing anxiety and stress. The invention offers one point of a hook & loop closure attachment, oriented in front of the individual for swift, independent removal.

The invention is also based, in part, on the insight that what is needed is a simpler, less invasive, pressure applying garment that is easier to apply to the mammalian subject (be it animal or human) offering a calming benefit without with an alternative system that avoids overheating the subject, is easy to put on and remove, and which doesn't restrict the subject's ability to move.

The invention is also based, in part, on the insight that certain textured elastic materials, such as the “sharkskin” textured neoprene rubber skin finishes produced by Macro Neoprene, Macro International Co, Irvine California, when applied around a subject's chest or trunk with appropriate force, tends to remain in place without sliding. Such materials, when appropriately configured, can form the basis of a simpler type of therapeutic pressure straps that can be better tolerated by the subject.

Examples of adequate sharkskin textures include anti-slip sharkskin neoprene with texture depth between 2 and 4 mm deep, and

Put alternatively, in some embodiments, the invention may be a therapeutic pressure strap for an animal user. The strap comprises a strip of material 1″ to 48″ wide and 8″ to 120″ long. The material comprises a stretchy material in which a tab of Velcro hook and loops can be fastened to one end so as to affix strap around animal trunk. The strap will be fastened in a snug manner to create the sense of deep pressure resulting in a sense of calm for the animal. The pressure strap is configured so that in cases of animal anxiety the strap may be donned around the animal's trunk to receive deep prolonged snug pressure (not tight) which modulates tactile sensation and calms the nervous system. This calming pressure mimics the same tactile input of humans petting their dogs, or when puppies pile-up on each other to take a nap. The animal is better able to tolerate and adapt to anxiety provoking events or manage generalized anxiety. The straps can be configured in varying widths, lengths and thicknesses based on the size, weight and species of the animal.

### BRIEF DESCRIPTION OF THE DRAWINGS

FIG. 1A shows an overview of the device for an animal as packaged for retail sale.

FIG. 1B shows the front of the package, showing a better view of the instructions for use.

FIG. 2 shows an overview of the back of the device as packaged for retail sale.

FIG. 3 shows a close up of the front end of the device.

FIG. 4 shows a side view of the front end of the device.

FIG. 5 shows a close up showing how in use, the device will form a loop around the subject (animal/human) such that the Velcro (hook) inner layer can form an attachment with the Velcro (loop) outer layer at any arbitrary section of the device.

FIG. 6 shows how the device may be affixed around the torso of an animal.

### DETAILED DESCRIPTION OF THE INVENTION

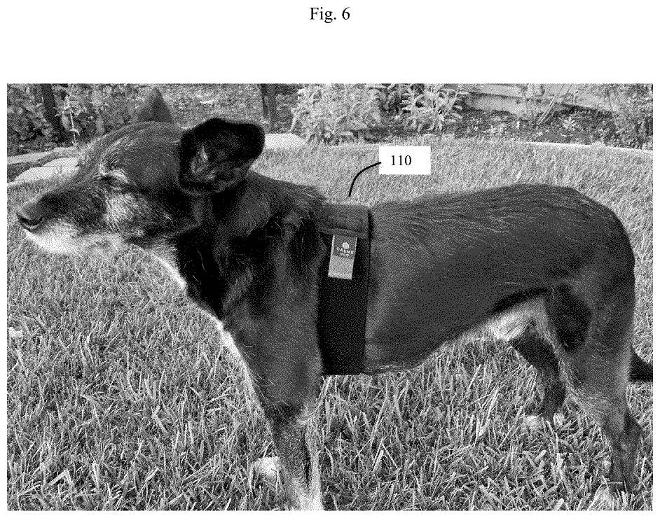

In some embodiments, the invention may be a method or system for calming a mammal (this may be an animal such as a dog, or a human). Expressing the invention in methods form, the invention comprises applying a strap device (100) around a trunk or chest of a mammal (see FIG. 6). This strap is typically linear—that is, a long, straight length of material, resembling a belt, without side branches.

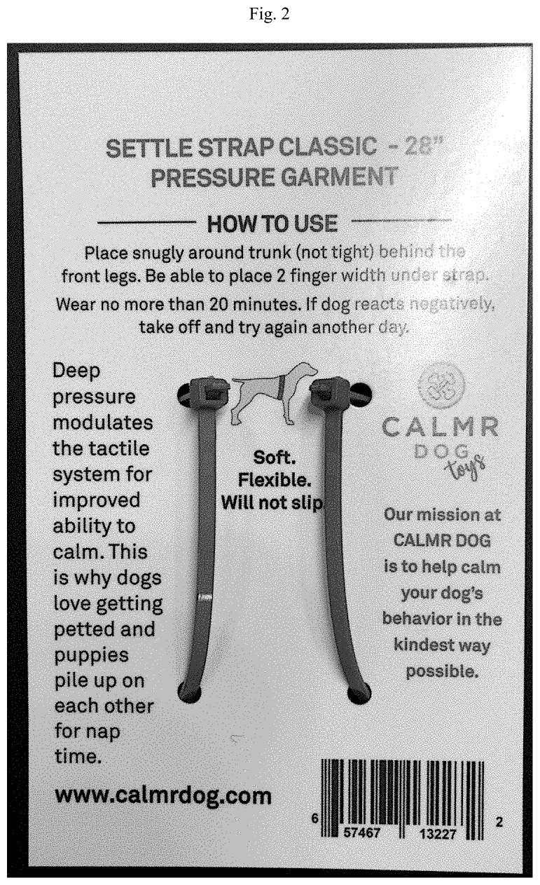

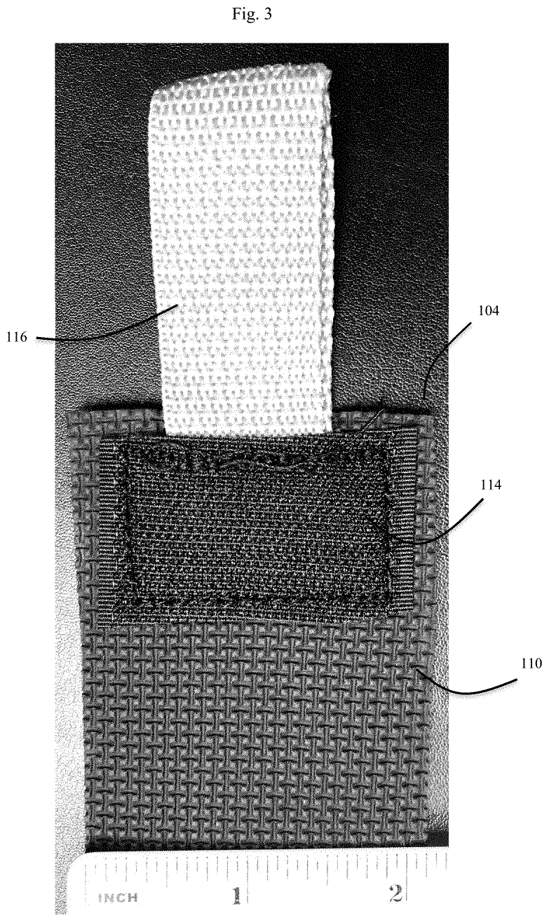

The strap device typically comprises a substantially flat laminated material (FIG. 4, 100). The first side of this laminated material (102) typically comprises a textured elastic material (FIG. 3 and FIG. 4, 110) with the texture side facing out, so that this textured side is configured to resist slippage when applied to the subject mammal's skin or fur. The second side (other side) (FIG. 4, 112) of this laminated material comprises one portion of a hook and loop fastening system such as Velcro. This portion can be either the hook portion (hook material) of the hook and loop fastening system, or it can be the loop portion of this hook and loop binding or fastening system. (In FIG. 4, 112 comprises the loop portion). The front end of the strap device is shown in FIG. 4, 104).

A portion (114) of the front end (104) of the strap device, often mounted above the textured material side (110) (thus creating a short “binding patch” section of a three-layer laminate in this region with the textured material (110) in the middle), typically comprises a complementary section of either a hook material or a loop material this hook and loop binding system. This “binding patch” region is shown in FIG. 4, 114).

As a result, when the strap device is wound around the mammal (see FIG. 6), the short complementary hook and loop material in the “patch” section (114) binds to the hook and loop material in the second (main) side of the laminated material (112) so as to form a reversible bond between the patch (114) at the front of the strap (104), and the the second complementary hook and loop side of the strap (112), usually at or near the rear of the strap.

More specifically, in some embodiments, a second side of the laminated material (112) can comprise a loop material of the hook and loop binding system. In such embodiments, the front end (104) of the strap device (100) can further comprise a complementary section of hook material (114) of this hook and loop binding system.

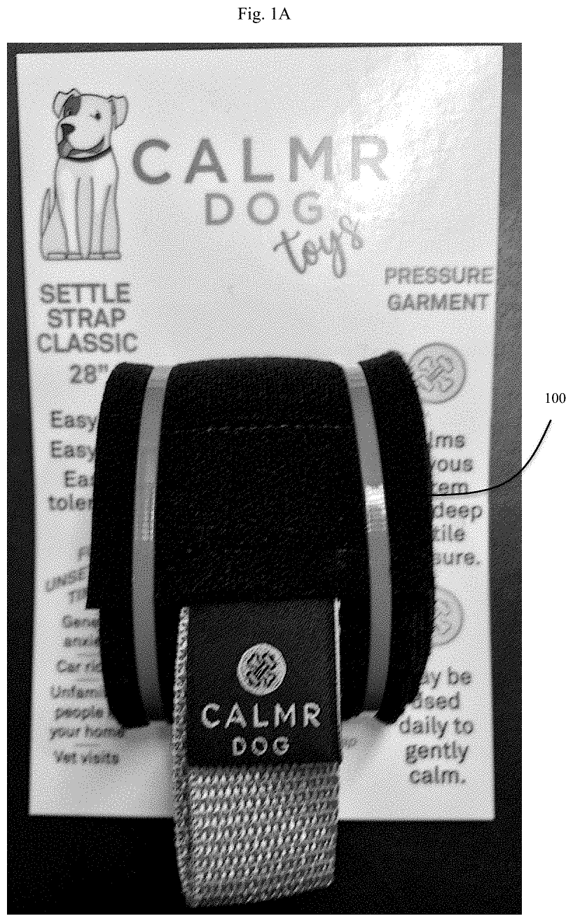

Put alternatively, the invention may be a therapeutic pressure strap or garment for an animal/human user (see FIG. 1A and FIG. 6 (100). The strap, belt, or garment comprises a strip of material 1″ to 24″ wide (typically about 1-3 inches wide for a dog-sized user) and 8″ to 120″ long (preferably around 28″ to 52″ inches long for dog/human sized users).

FIG. 3 shows a close up of the front end of the device (104), showing the strap, a “patch” section of Velcro (hook and loop) material (114), and a close up of a textured neoprene material (110) that is useful to help prevent the device from slipping off of the animal's skin or fur. A ruler calibrated in inches is also shown for scale. In this example, the device is a bit over two inches wide. Often the strap device will have a width between 1.5 inches and 3 inches, and a length between 20 inches and 40 inches.

As can be seen in FIG. 3, FIG. 4 and elsewhere, the front end of the strap device (104) will often further comprise any of a pull tap or finger loop (116). This pull tap or loop is optional, but is often useful to make it easier to apply the device and remove the device. This pull tap or finger loop is often affixed to the front of the device by sewing or other robust attachment method.

In some embodiments, this textured elastic material (110) can comprise a two-dimensional array of hills and valleys, with a texture height between 1 and 4 mm between a bottom of the valleys and a top of the hills. These hills and valleys may be configured at a density of between 25 to 400 hills per square inch. In some embodiments, this textured material may comprise a sharkskin textured neoprene material.

FIG. 4 shows a close up of the device, showing how the material comprises a multiple layer laminated belt. As previously discussed, one side (110) will typically comprise a stretchy material such as neoprene, preferably textured neoprene (see FIG. 3). The other side (112) will typically comprise a Velcro (hook and loop) like material, such as a Velcro loop material. The two sides can be laminated together by glue, stitching, heat, or other process.

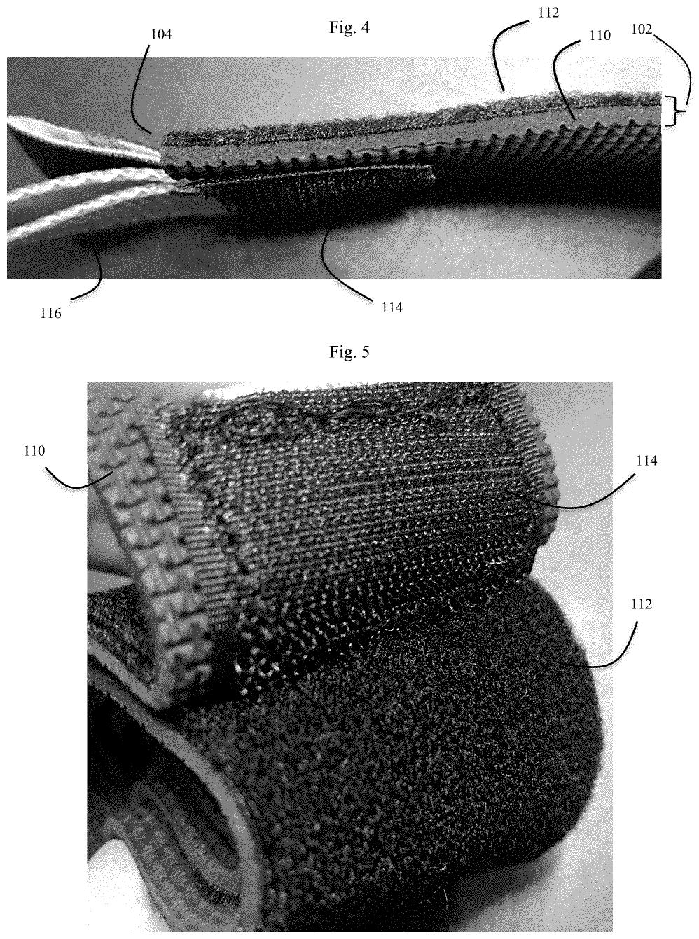

In a preferred embodiment, a small segment of the complementary Velcro (hook and loop) material (114) will be glued or stitched on top of the of textured material (110) so as to form a small binding patch or region (114), near the front end of the belt device (104), that will bind to the complementary hook and loop material (112) when the belt is wound around the animal/human (see FIG. 5 and FIG. 6).

More specifically, FIG. 4 shows a side view of the front end of the device (104), showing the small binding patch (loop) portion of the hook and loop material (114), the sharkskin neoprene layer (110), and the loop portion of the hook and loop outer layer (112). In use, the sharkskin neoprene layer (110) is laminated, glued, or sewn together with the Velcro (loop) outer layer (112) to provide a continually laminated device.

FIG. 5 shows a close up of how the device (100) can form a loop around the subject (animal/human) and be fastened. The “binding patch” portion of the device (114), here comprising a “hook” portion of hook and loop material (laminated on top of textured elastic material 110) is shown forming an attachment with the complementary hook and loop (here loop material 112) on the other side of the device. This enables the user to apply the desired pressure (tension) to the device, and also allow the device to fit a broad range of different subject sizes and types.

FIG. 6 shows how the device (100) may be affixed around the trunk or torso of an animal, here a dog. Note that the device can apply calming pressure without otherwise encumbering or disturbing the subject. Humans, for example, can wear this device discreetly under clothing.

In a preferred embodiment, the material is configured so that the user may, independently or not, wrap the belt device around the animal/human (usually the torso/trunk) so as to apply gentle pressure to the animal/human trunk, but otherwise not greatly encumber the individual. The material is preferably configured to be easy to cut with scissors so that the user can trim off any excess length as needed.

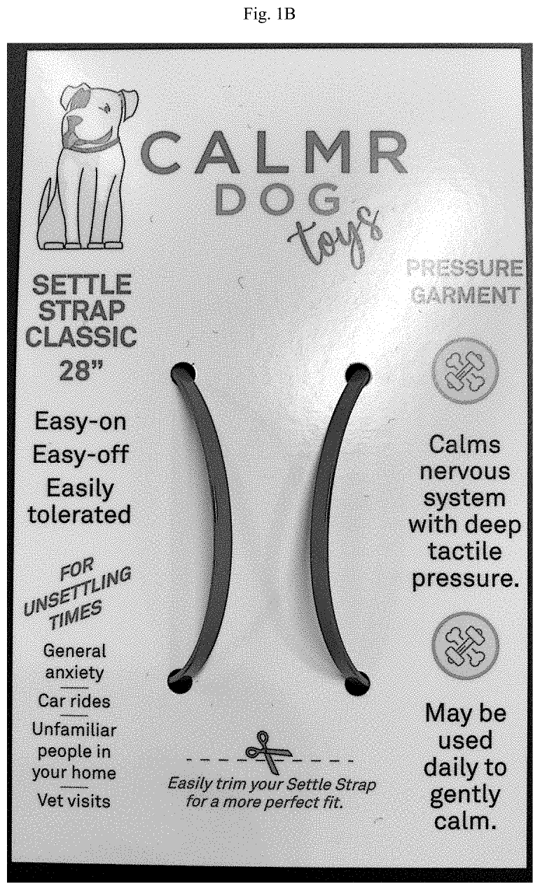

The instructions shown in FIG. 1B and FIG. 2 are reproduced below.

These instructions say: SETTLE STRAP CLASSIC 28″ Easy-on, Easy-off, easily tolerated for unsettling times General anxiety, Car rides Unfamiliar people in your home Vet visits Pressure Garment Calms nervous system with deep tactile pressure. May be used to gently calm. Easily trim your Settle Strap for a more perfect fit.

FIG. 2 shows the other side of the instructions, which say: SETTLE STRAP CLASSIC— 28″ PRESSURE GARMENT HOW TO USE

Place snugly around trunk (not tight) behind the front legs. Be able to place 2 finger width under strap. Wear no more than 20 minutes. If dog reacts negatively, take off and try again another day.

Deep pressure modulates the tactile system for improved ability to calm. This is why dogs love getting petted and puppies pile up on each other for nap time.

Soft. Flexible. Will not slip.

CALM DOG toys. Our mission at CALM DOG is to help calm your dog's behavior in the kindest way possible.

In a preferred embodiment, the strap is fastened in a snug manner to create a sense of deep pressure resulting in a sense of calm for the subject. The pressure garment is configured so that in cases of anxiety the garment may be donned around the animal/human trunk to provide deep prolonged snug pressure (not tight) which modulates tactile sensation and calms the nervous system.

In some embodiments, an optional pressure indicator may be incorporated into the belt device. This can help the user achieve a uniform pressure between uses. Various types of pressure indicator can be used, including materials that change color according to pressure, that deform according to pressure, or even (for high end units) pressure indicators with Bluetooth transceivers that interface with smartphones or other types of devices.

Without being bound by any theory, the applicant speculates that the device operates to provide a calming pressure that mimics the same tactile input that bonding with other animals, or humans (e.g., petting, pressure provided from litter-mates, swaddling or hugging) can produce. The animal is better able to tolerate and adapt to anxiety provoking events or manage generalized anxiety. The straps can be configured in varying widths, lengths and thicknesses based on the size, weight and species of the animal.

First public display: The invention was first displayed to the public on March 23 to 25, 2022 at the Global Pet Expo, Orange County Convention Center, Orlando Florida.

First sale: First sales of the invention occurred on Mar. 31, 2022.

First publication: The invention was first displayed on the applicant's internet website, www.calmrdog.com/on Mar. 30, 2022.

Velcro® is a trademark of Velcro IP Holdings, LLC.

1. A method of calming a mammal, said method comprising:
applying a strap device around a trunk or chest of said mammal, said strap device comprising a laminated material;
a first side of said laminated material comprising a textured elastic material configured to resist slippage when applied to said mammal's skin or fur;
a second side of said laminated material comprising either a hook material or a loop material of a hook and loop binding system;
a front end of said strap device further comprising a complementary section of either a hook material or a loop material of said hook and loop binding system, such that when said strap device is wound around said mammal, said complementary section binds to said second side of said laminated material so as to form a reversible bond with said second side.

2. The method of claim 1, wherein said textured elastic comprises a two-dimensional array of hills and valleys, with a texture height between 1 and 4 mm between a bottom of said valleys and a top of said hills, said hills and valleys configured at a density of between 25 to 400 hills per square inch.

3. The method of claim 1, wherein said elastic material comprises a sharkskin textured neoprene material.

4. The method of claim 1, wherein said laminated material is configured to be cuttable by scissors.

5. The method of claim 1, wherein said strap device has a width between 1.5 inches and 3 inches, and wherein said strap device has a length between 20 inches and 40 inches.

6. The method of claim 1, wherein said front end of said strap device further comprises any of a pull tap or finger loop.

7. The method of claim 1, wherein said second side of said laminated material comprises a loop material of said hook and loop binding system.

8. The method of claim 7, wherein said front end of said strap device further comprises a complementary section of hook material of said hook and loop binding system.

9. The method of claim 1, wherein said mammal comprises any of an animal mammal or a human.

10. The method of claim 1, wherein said strap device comprises a flat, linear, non-branching belt.

11. A strap device for a mammal, said strap device comprising:
a flat laminated material comprising a first side, a second side, a front end and a back end;
said first side of said laminated material comprising a textured elastic material configured to resist slippage when applied to said mammal's skin or fur;
said second side of said laminated material comprising any of a hook material or a loop material of a hook and loop binding system;
said front end of said strap device further comprising a complementary section of either said hook material or said loop material of said hook and loop binding system;
said strap device configured so that when said strap device is wound around a trunk or chest of said mammal, said complementary section binds to said second side of said laminated material so as to form a reversible bond with said second side.

12. The device of claim 11, wherein said textured elastic comprises a two-dimensional array of hills and valleys, with a texture height between 1 and 4 mm between a bottom of said valleys and a top of said hills, said hills and valleys configured at a density of between 25 to 400 hills per square inch.

13. The device of claim 11, wherein said elastic material comprises a sharkskin textured neoprene material.

14. The device of claim 11, wherein said laminated material is configured to be cuttable by scissors.

15. The device of claim 11, wherein said strap device has a width between 1.5 inches and 3 inches, and wherein said strap device has a length between 20 inches and 40 inches.

16. The device of claim 11, wherein said second side of said laminated material comprises a loop material of said hook and loop binding system.

17. The device of claim 16, wherein said front end of said strap device further comprises a complementary section of hook material of said hook and loop binding system.

18. The device of claim 11, wherein said front end of said strap device further comprises any of a pull tap or finger loop.

19. The device of claim 11, wherein said mammal comprises any of an animal mammal or a human.

20. The device of claim 1, wherein said strap device comprises a flat, linear, non-branching belt.
